(19) United States
(12) Patent Application Publication
(10) Pub. No.: US 2022/0172835 A1
(43) Pub. Date: Jun. 2, 2022

(54) OUTPUT DEVICE FOR OUTPUTTING A MEASURED VALUE PROGRESSION

(71) Applicant: Drägerwerk AG & Co. KGaA, Lubeck (DE)

(72) Inventors: Michael GÖMANN, Lubeck (DE); Christian WEBER-LEHN, Lubeck (DE)

(21) Appl. No.: 17/538,253

(22) Filed: Nov. 30, 2021

(30) Foreign Application Priority Data
Dec. 2, 2020 (DE) 10 2020 131 947.9

(51) Int. Cl. G16H 40/67; A61B 5/00

(52) U.S. Cl. CPC G16H 40/67; A61B 5/7221; A61B 5/4848; A61B 5/746; A61B 5/7475; A61B 5/743

(57) ABSTRACT

An output device (100) outputs a measured value temporal course (122) of a medical measured value (107) and includes a receiving unit (110), an output control unit (120) and a display unit (130). The receiving unit receives sensor data (114) indicating the measured value temporal course and signal quality data (116) indicating a signal quality progression. The output control unit (120) determines a graph (124), providing an output signal (128) indicating a measured value curve (125), based on the sensor data and the signal quality data, with a curve spread (123) indicative of signal quality present corresponding to the signal quality data at the time of the measured value curve. The display unit receives the output signal and graphically outputs the graph with the measured value curve and with the corresponding curve spread. The curve spread is displayed as a corresponding spread (126) of the measured value time curve.

### CROSS REFERENCE TO RELATED APPLICATIONS

This application claims the benefit of priority under 35 U.S.C. § 119 of German Application 10 2020 131 947.9, filed Dec. 2, 2020, the entire contents of which are incorporated herein by reference.

### TECHNICAL FIELD

The present invention pertains to an output device for outputting a measured value progression or measured value curve (time curve or temporal course) of a medical measured value. The present invention pertains, furthermore, to an output system for outputting a measured value progression or time curve of a medical measured value, to a process for outputting such a measured value progression or time curve and to a computer program with a program code for carrying out such a process.

### TECHNICAL BACKGROUND

It is generally known that measured value curves are outputted in a medical context in order to make it possible to detect changes in the physiological condition of a patient during the patient's treatment and to take them into consideration for the further treatment. Furthermore, it is known that parameters can be determined from a measured value curve manually or in an automated manner. Such a parameter may be useful for the clinical assessment of the current measured value of the measured value curve. Furthermore, a current status of the patient can be detected thereby rapidly.

It is known that the output of the measured value curve may be carried out at a distance from the medical device proper. For example, the medical device may be connected via a network to a corresponding output device. Thus, German Patent Application DE 10 2019 003 995 describes a display unit, which is connected to a medical device in order to output corresponding measured data graphically as a separate device.

### SUMMARY

An object of the present invention is to provide an improved output device, especially an output device with a signal quality that can be detected in an especially simple manner.

According to a first aspect of the present invention, an output device for outputting a measured value progression or time curve (temporal course) of a medical measured value with a receiving unit, with an output control unit and with a display unit is provided.

The receiving unit is configured to receive sensor data and signal quality data via at least one received signal and to make it available for the further processing by the output device, wherein the sensor data indicate the measured value progression or time curve of the medical measured value, and wherein the signal quality data indicate a signal quality time curve associated with the temporal course of the measured values.

The output control unit is configured to determine a graph to be outputted with a measured value curve on the basis of the sensor data and the signal quality data and to make it available via an output signal, wherein the measured value curve indicates the temporal course of the measured values and a curve spread of the measured value curve, and wherein the measured value curve indicates, due to its position and structure within the graph to be outputted, the temporal course of the measured values of the medical measured value. The respective curve spread associated with a respective time of the measured value curve is (comprises) a value indicative of the signal quality present corresponding to the signal quality data at this time of the measured value curve.

The display unit is configured to receive the output signal and to output the graph to be outputted graphically on a display of the display unit with the measured value curve and with the corresponding curve spread. The curve spread being displayed as a corresponding spread of the temporal course of the measured values.

It was found within the framework of the present invention that together with the measured value curve, additional information can be outputted for evaluating this measured value curve. Thus, an output of a spread of the measured value curve is provided together with the measured value curve in order to also output information concerning the currently present signal quality directly with the measured value curve to a user of the output device. In particular, the output of two different visual areas for the output of this information on a display is advantageously avoided hereby.

Even though it is known that a number or a separated graphic element, e.g., a bar, can be displayed in order to display a current signal quality, the solution according to the present invention has, by contrast, the advantage that no additional cognitive activity is necessary besides the detection of the current measured value curve to directly perceive the curve spread and hence information concerning the currently present signal quality.

Further, the display of the curve spread for the measured value curve also makes it possible to estimate the accuracy of earlier displayed values, in which an especially low or especially high signal quality was possibly present. Thus, the output device according to the present invention supports an especially reliable analysis of the medical measured values displayed.

The direct association of sensor data and signal quality data makes it possible to display a chronological sequence of spreads of the measured value curve. As a result, it becomes possible to directly detect a tendency towards especially high or especially low signal quality, which tendency is possibly becoming established, so that changes in a communication connection to the corresponding medical system can be immediately detected and taken into consideration in the analysis of the corresponding measured values. In addition, physical changes in the communication connection, e.g., a changeover from wireless connection to cable-based connection and/or a change of the current location of the output device, can be suggested.

That sensor data and signal quality data on at least one received signal are received means that they can be received according to the present invention within a single signal, by two separate signals and/or by a plurality of signals, e.g., by a sequence of separate signals.

The graph to be outputted makes it possible according to the present invention on the basis of its structure to provide information about a quantitative property of the measured value currently present. The graph may comprise for this a coordinate system or comparable graphic means for illustrating a value or a current area of the currently present measured value.

A spread is a visual illustration of an area, which is broader than a mere dot or a mere line, which indicates the currently present measured value and/or the currently present measured value curve.

The measured value curve may indicate the measured value curve continuously or in the form of discrete measured values, e.g., discrete dots within the graph.

The units of the output device according to the present invention may be arranged at least partially at spaced locations from one another, especially partially in separate housings. The units are preferably arranged in a common housing. In particular, the units may be configured especially by a common processor, and the units are separated from one another at least at the software level.

Preferred embodiments of the output device according to the present invention will be described below.

In an advantageous embodiment, the spread is a symmetrical spread of the measured value curve around a measured value indicated by the sensor data as a center of the spread. A low signal quality leads in this embodiment to the assumption of a symmetrical error around the measured value to be outputted. As a result, the signal quality can be detected especially rapidly because it is not necessary for the user to compare two measured value ranges around the measured value proper with one another. The provision of a symmetrical spread can advantageously lead to a lower calculation time by the output device than for the case in which two different spread areas must be determined.

In an especially preferred embodiment, the areas of the measured value curve that indicate the measured value curve are displayed with more contrast than the corresponding spread. As a result, the user of the output device according to the present invention can detect the respective measured value within the measured value curve especially rapidly and reliably without having to analyze the structure of the measured value curve. The measured value curve is especially preferably displayed in one color, for example, in black on a white background and white in case of a dark background. The spread in this case preferably has different contrast intensities starting from the measured value curve. Thus, an outer area of the spread is shown preferably almost in the color of the background of the display.

In an advantageous embodiment, the spread is displayed as a continuously extending spread around areas of the measured value curve, which areas indicate the measured value curve. A continuously extending spread is preferably a spread whose outer area has a continuous, especially continuously differentiable edge, i.e., especially an edge without jumps or the like. As a result, a signal quality is unambiguously assigned to each time within the measured value curve. In an alternative embodiment, the spread is provided as a discontinuous spread, especially as a spread displayed discretely for individual measured values at individual times.

The spread is especially preferably inversely proportional to the signal quality present at this time corresponding to the signal quality data. The spread is especially understandable in this embodiment because a high signal quality leads to a small spread and hence to an especially narrow spread area around the measured measured value of the measured value curve. The spread can thus be interpreted intuitively as a possible measurement error, which is especially great when the signal quality is especially low. This is not, in fact, a measurement error according to the present invention, but a signal transmission error. In one embodiment according to the present invention, the spread does, moreover, depend on a predefined systematic measurement error of the device that has determined the measured values. Such a systematic measurement error may be stored in a corresponding memory module. In an alternative or additional embodiment according to the present invention, a temporary measurement error known on the basis of a current treatment of a patient is taken into consideration in the determination of a value) of the curve spread. Such a temporary measurement error may be present, for example, due to an activity that displays a burden for the circulation.

In a preferred embodiment, the output control unit is configured to take into consideration a time distance to the last occurring measurement of a corresponding medical measured value in the determination of the value of the curve spread. A determined measured value may thus be burdened with a considerable inaccuracy already after a few seconds for a greatly fluctuating value, because this measured value possibly has only a low validity concerning the currently actually present measured value.

In a preferred embodiment, the output control unit is further configured to access a memory module with a stored plurality of graphic displays for the graph to be outputted. As a result, a user-defined setting of the graphic display of the measured value curve is possible. The stored plurality of graphic displays may comprise different colors and/or color combinations for the graph to be outputted. Furthermore, the different graphic displays may comprise different displays for the curve spread.

In an advantageous embodiment, the receiving unit is further configured to receive alarm generation data, wherein the alarm generation data indicate at least one alarm generation limit for the medical measured value and wherein the output control unit is further configured to determine the graph to be outputted with the at least one alarm generation limit and to make it available via the output signal. Due to the preferably visual output of the alarm generation limit, a user can detect rapidly whether the measured values of the measured value curve or at least the curve spread reaches the vicinity of the alarm generation limit. In particular, it can be estimated whether an especially great curve spread, i.e., an especially low signal quality, was present when the alarm generation limit was exceeded.

According to a second aspect of the present invention, an output system for outputting a measured value curve of a medical measured value is provided to accomplish the above-mentioned object. The output system in this case comprises an output device according to at least one of the above embodiments and a data acquisition device. The data acquisition device is configured here to detect the sensor data via a received sensor signal, to determine the signal quality of the sensor signal and to associate the corresponding signal quality data, especially the corresponding current signal quality data, to the sensor data. Furthermore, the data acquisition device is configured to output the sensor data and the associated signal quality data to the output device. The output may take place now directly to the output device, especially in the form of the received signal, or indirectly via an additional device. The additional device may be, for example, a device that translates a signal from a first signal protocol into a second signal protocol. As a result, a compatibility may be possible between different devices and/or between devices of different manufacturers in an especially advantageous manner.

The determined signal quality is preferably based on the signal-to-noise ratio of the received sensor signal. As an alternative or in addition, the determination of the signal quality may be carried out, for example, via the so-called SINAD value (signal-to-interference ratio including noise and distortion). As an alternative or in addition, a dynamic range of the recorded data can be taken into consideration, for example, by taking into account the graining of the measurement, e.g., the pixel width, the sensor resolution and the like.

In an especially advantageous embodiment of the output system, the data acquisition device is configured to determine a correlation between the sensor data and the associated signal quality data over time and to output it to the output device, the output device being further configured to determine the spread of the measured value curve on the basis of the correlation. If, for example, the signal quality is always at the same level fully independently from the sensor data, a time distance between times at which the curve spread is determined can be increased, because no change can be expected in the curve spread. As an alternative or in addition, a type of sensor data may have a close correlation with the associated signal quality, so that a similar signal quality is always present for a concrete type of sensor data. The type of the sensor data may be a data type, a measured value type, a device type for the determination of the measured value or the like.

The output system is configured in a preferred embodiment to provide a communication connection with an external alarm generation system and to provide a warning signal if a currently determined signal quality of the sensor signal is below a predefined limit value. A user of the alarm generation system as well as a user of the output system are informed in this embodiment that the currently determined signal quality is below the predefined limit value. As a result, a response of the user, for example, a repair of the corresponding connection or a re-establishing of the connection can be initiated. In a variant of this embodiment, the output system is further configured to provide a warning signal if a measured value within the measured value curve to be outputted exceeds a predefined threshold value. The provision of the warning signal preferably takes place via the communication connection through the external alarm generation system. As an alternative or in addition, the warning signal may be outputted additionally or exclusively by the output system optically or acoustically to a user of the output system.

In an advantageous embodiment, the output system has, furthermore, a user interface, which is configured to receive a user input, wherein the user input pertains to an operating mode of the output system, to a selection of a graphic display for the graph to be outputted, especially for the spread of the temporal course of the measured values to be displayed, from a stored plurality of graphic displays and/or a selection of a determination rule for the determination of the signal quality from a stored plurality of determination rules. The operating mode of the output system, which operating mode is to be selected, may comprise, for example, an activation or a deactivation of the output system. The graphic displays can be adapted in this embodiment to the preferences of the user of the output system.

According to a third aspect of the present invention, a process for outputting a temporal course of the measured values of a medical measured value is provided for accomplishing the above-mentioned object. The process according to the present invention has the following steps:
receipt and provision of sensor data and signal quality data, wherein the sensor data indicate the temporal course of the measured values of the medical measured value, and wherein the signal quality data indicate a signal quality time curve associated with the temporal course of the measured values;
determination of a graph to be outputted with a measured value curve based on the sensor data and on the signal quality data and provision of a corresponding output signal, wherein the measured value curve indicates the temporal course of the measured values and a curve spread of the temporal course of the measured values, and wherein the measured value curve indicates by its position and structure within the graph to be outputted the temporal course of the measured values of the medical measured value, and wherein the respective curve spread associated with a time of the measured value curve is a quality value indicative of the signal quality present at this time of the measured value curve corresponding to the signal quality data; and
receipt of the output signal and graphic output of the graph to be outputted with the measured value curve, wherein the curve spread is displayed as the corresponding spread of the temporal course of the measured values.

The steps of the process according to the present invention are carried out in the order shown. Thus, the sensor data and the signal quality data are received only before the output signal with the measured value curve is determined and then outputted graphically based on these sensor data and signal quality data.

The process steps are carried out at least nearly in real time, so that there are less than 5 sec, preferably less than 2 sec and especially preferably less than 1 sec between the receipt of the data and the graphic output of the graph to be outputted. As a result, a user of the process according to the present invention can detect the current state of the patient and the current signal quality via the graphic output especially rapidly.

The steps of the process according to the present invention are preferably carried out by a single device. As an alternative, at least one of the process steps may be carried out on a device located at a distance.

According to a fourth aspect of the present invention, a computer program with a program code for carrying out a process according to the third aspect of the present invention when the program code is executed on a processor or on a programmable hardware component is provided for accomplishing the above-mentioned object. A plurality of steps of the process according to the present invention are carried out by a shared computer, by a shared processor or by a shared programmable hardware component. The individual steps are preferably separated from one another by corresponding software blocks at least at the software level. All steps of the process according to the present invention are especially preferably carried out on a shared computer, on a shared processor or on a shared programmable hardware component.

The present invention shall be explained now in more detail on the basis of advantageous embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of an output device according to a first aspect of the present invention;

FIG. 2 is a schematic view of a measured value curve of an output device according to the first aspect of the present invention, wherein a discontinuous spread is shown;

FIG. 3 is a schematic view of a measured value curve of an output device according to the first aspect of the present invention, wherein a continuous spread is shown;

FIG. 4 is a schematic view of a measured value curve of an output device according to the first aspect of the present invention, wherein a discrete spread is shown;

FIG. 5 is a schematic view of a second exemplary embodiment of the output device according to the first aspect of the present invention;

FIG. 6 is a schematic view of a first exemplary embodiment of an output system according to the second aspect of the present invention;

FIG. 7 is a schematic view of a second exemplary embodiment of the output system according to the second aspect of the present invention; and

FIG. 8 is a flow chart of an exemplary embodiment of a process according to a third aspect of the present invention.

### DESCRIPTION OF PREFERRED EMBODIMENTS

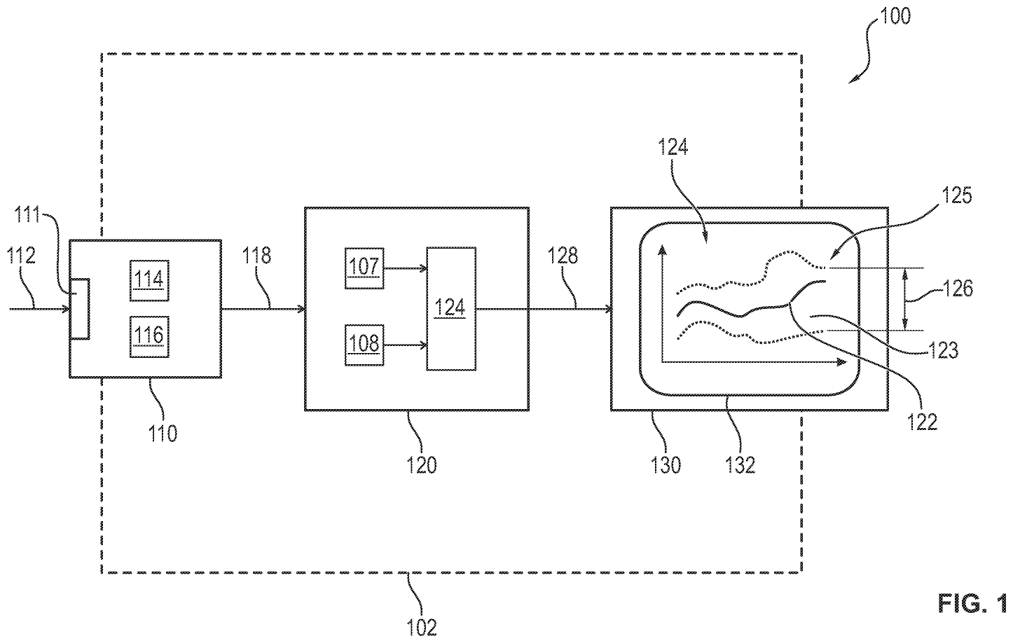

Referring to the drawings, FIG. 1 shows a schematic view of a first exemplary embodiment of an output device 100 according to a first aspect of the present invention.

The output device 100 is configured to output a temporal course of the measured values (measured value progression or time curve) 122 of a medical measured value 107. The output device 100 has for this purpose a receiving unit 110, an output control device 120 and a display unit 130.

The receiving unit 110 is configured to receive sensor data 114 and signal quality data 116 via at least one received signal 112 and to make this data available for further processing by the output device 100. The provision is carried out in the exemplary embodiment shown via a processed signal 118. The sensor data 114 indicate the temporal course of the measured values 122 of the medical measured value 107, and the signal quality data 116 indicate a signal quality time curve associated with the temporal course of the measured values 122. The sensor data 114 and the signal quality data 116 are provided in this case by an external device, not shown, with which the receiving unit 110 communicates in a cable-based manner. The receiving unit 110 has for this purpose a receiving interface 111 for receiving the received signal 112. The sensor data 114 and the signal quality data 116 are provided in this case by a single received signal 112, which transports the corresponding data. As an alternative or in addition, a plurality of received signals may be provided for receiving the data or other information.

The output control unit 120 is configured to determine a graph 124 to be outputted with a measured value curve (measured value progression or temporal course) 125 on the basis of the sensor data 114 and the signal data 116 and to make the graph 124 available via an output signal 128. The measured value curve 125 indicates the temporal course of the measured values 122 and a curve spread 123 of the temporal course of the measured values 122. The measured value curve 125 indicates by its position and structure within the graph 124 to be outputted the temporal course of the measured values 122 of the medical measured value 107. As is seen from the exemplary embodiment shown, this can be brought about via a position within a coordinate system. This is brought about in other exemplary embodiments shown via marks, especially separate marks, which indicate a magnitude of corresponding measured values and/or of the corresponding time. The curve spread 123 associated with a time of the measured value curve 125 is a quality value indicative of the signal quality 108, which is present corresponding to this time of the measured value curve 125.

As is shown schematically in FIG. 1, values for the medical measured value 107 and the signal quality 108 are determined from the sensor data 114 and from the signal quality data 116 and are combined into the graph 124 to be outputted.

The display unit 130 is configured to receive the corresponding output signal 128 and to graphically output the graph 124 to be outputted with the measured value curve 125 and with the corresponding curve spread 123 on a display 132 of the display unit 130. The curve spread 123 is displayed as a corresponding spread 126 of the temporal course of the measured values 122.

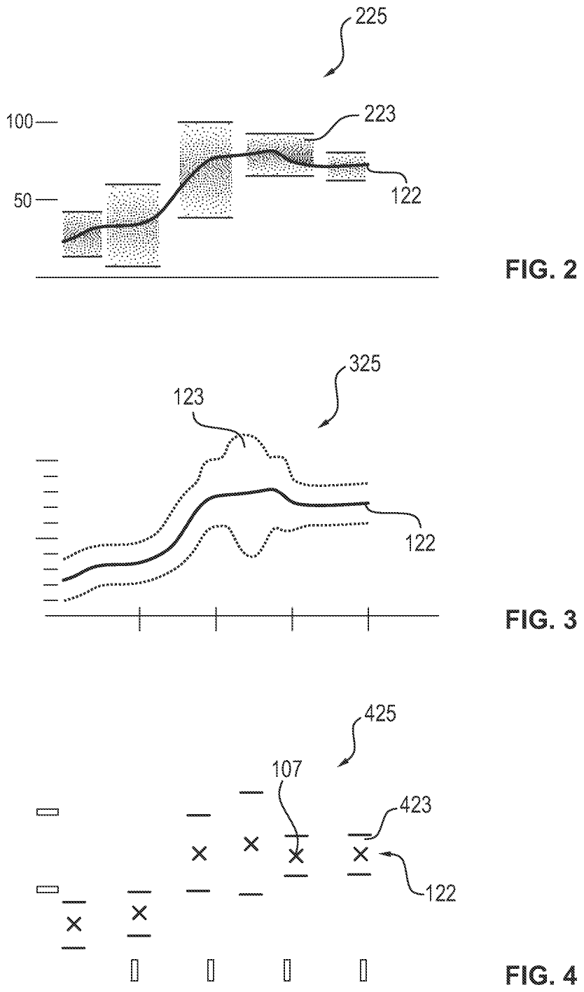

This spread 126 of the temporal course of the measured values 122 may be carried out in different ways according to the present invention. An asymmetric spread starting from the temporal course of the measured values 122 is shown in the exemplary embodiment shown. Symmetrical spreads of the corresponding measured value curve are shown within the framework of FIGS. 2 through 4.

The temporal course of the measured values 122 is displayed in the exemplary embodiment shown by a solid, high-contrast line with a higher contrast than an edge area of the curve spread 123 shown, which is suggested by means of a dotted display. The spread 126 is a continuously extending spread around the temporal course of the measured values 122 and hence around the areas of the measured value curve 125 indicating the temporal course of the measured values 122, namely, it is displayed here by the solid line. The graph 124 can be seen in the exemplary embodiment shown as a graph by means of an X axis and a Y axis. The X axis describes here a time interval, whereas the Y axis depends on the medical measured value 107 being investigated.

The spread is inversely proportional to the signal quality 108 present at this corresponding time of the spread corresponding to the signal quality data 116.

The signal quality 108 is displayed in the exemplary embodiment being shown by values that are based on the signal-to-noise ratio of a previously received sensor signal. This ratio was determined by an external device, not shown, and is outputted via the receiving signal 112 to the output device 100 according to the present invention.

The units of the output device 100 according to the present invention may be arranged, as is shown in the exemplary embodiments shown, in a common housing 102. As an alternative or in addition, individual units of the output device according to the present invention may be located at spaced locations from one another, especially in separate housings. A wireless communication is especially advantageous for units located at spaced locations from one another. Such a wireless communication may take place via a network, via Bluetooth, via BLE, via ZigBee or the like.

The units are separated from one another at least at the software level in the exemplary embodiment shown and are embodied by a shared processor, not shown,

FIGS. 2, 3 and 4 show a schematic display of a respective measured value curve 225, 325, 425 of an output device according to the first aspect of the present invention, wherein a discontinuous spread (FIG. 2), a continuous spread (FIG. 3) and a discrete spread (FIG. 4) are shown.

The measured value curves 122 are identical for the measured value curves 225, 325 and 425, so that the schematic displays shown differ only in the manner of display of the graph, of the measured value curve and in the size and in the manner of the display of the spread.

FIG. 2 shows a Y axis with only two different values in order to estimate the order of magnitude of the present measured values. The X-axis displays a time range. This time range is shown without marks of the like.

The temporal course of the measured values 122 is displayed as a solid line. The curve spread 223 is displayed symmetrically around a measured value indicated by the sensor data as a center of the spread. The extent of the spread depends on a single measured value within a predefined time interval. The spread resulting from this is displayed for the entire time interval. The interval arising from the spread for the magnitude of the measured value is displayed with high contrast in the area of the temporal course of the measured values 122 and it has a lower contrast towards the edges of the spread.

FIG. 3 shows a Y axis without values stated explicitly but with lines to display the order of magnitude of the currently present measured value. The X axis displays a time range. This time range can be optically recognized by individual marks at predefined time intervals.

The display of the curve spread 123 is carried out as is shown in FIG. 1 via a low-contrast display of an edge area of this spread. Both the temporal course of the measured values 122 and the course of the curve spread 123 are shown continuously, so that a signal quality to be associated with the measured value in question can be recognized for every individual measured value displayed.

FIG. 4 shows both along the Y axis and along the X axis only individual marks, which make a corresponding area of the time interval shown and of the measured value interval shown optically recognizable.

The display of the curve spread 423 is carried out in discrete steps corresponding to the display of the temporal course of the measured values 122, which is likewise visualized by discretely displayed measured values 107. A spread, which yields the curve spread 423 displayed, is thus associated with each measured value shown.

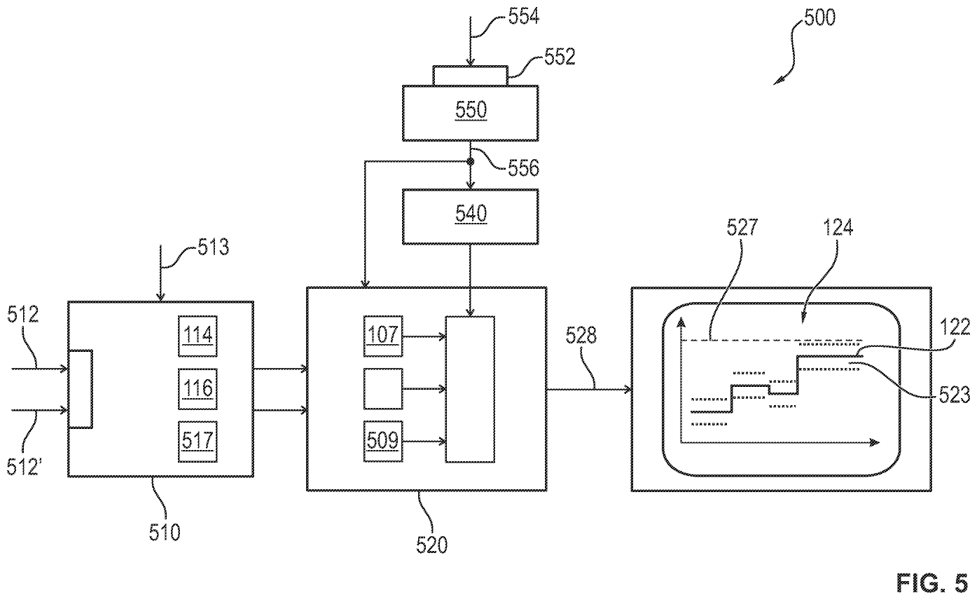

FIG. 5 shows a schematic view of a second exemplary embodiment of the output device 500 according to the first aspect of the present invention.

The output device 500 differs from the output device 100 shown in FIG. 1 in that the receiving unit 510 is configured to receive two received signals 512, 512′, wherein a received signal 512 indicates the sensor data 114 and the other received signal 512′ indicates the signal quality data 116. Different received signals, which indicate, for example, measured values analyzed at different times and/or by different external devices, are received by the receiving unit according to the present invention at time intervals in an alternative or additional exemplary embodiment.

In addition, the receiving unit 510 is configured to receive alarm generation data 517 via an alarm generation signal 513. The alarm generation data 517 indicating at least one alarm generation limit for the medical measured value 107. In addition, the output control unit 520 is configured to determine the at least one display of an alarm generation limit 509, which display is to be outputted and to make it available via the output signal 528. The alarm generation limit 509 is displayed as an alarm generation line 527 in the graph 124.

Furthermore, the output device 500 comprises a memory module 540, in which a plurality of graphic displays for the graph 124 to be outputted are stored. The memory module 540 is configured to output a currently predefined graphic display to the output control unit 520 in order for this to use the currently predefined graphic display for the determination of the graph 124 to be outputted.

Finally, the output device 500 comprises, contrary to the output device 100, an input unit 550, which is configured to receive a user input 554 via an input interface 552 and to output a corresponding input signal 556 to the memory module 540 and/or to the output control unit 520. The input signal 552 may indicate a selection of the currently predefined graphic display, which is to be used as a graphic display, it may indicate a current operating mode of the output device 500, e.g., an activation or deactivation, and/or it may indicate an activation or deactivation of the output of the alarm generation limit 509.

In one exemplary embodiment, not shown, the input signal may indicate a reception mode for the receiving unit according to the present invention and/or an output mode of the display unit according to the present invention.

The temporal course of the measured values 122 is displayed in this exemplary embodiment as a continuous course, which has a corresponding jump to this measured value with each new measured value. The curve spread 523 likewise has correspondingly a jump for each new discrete measured value.

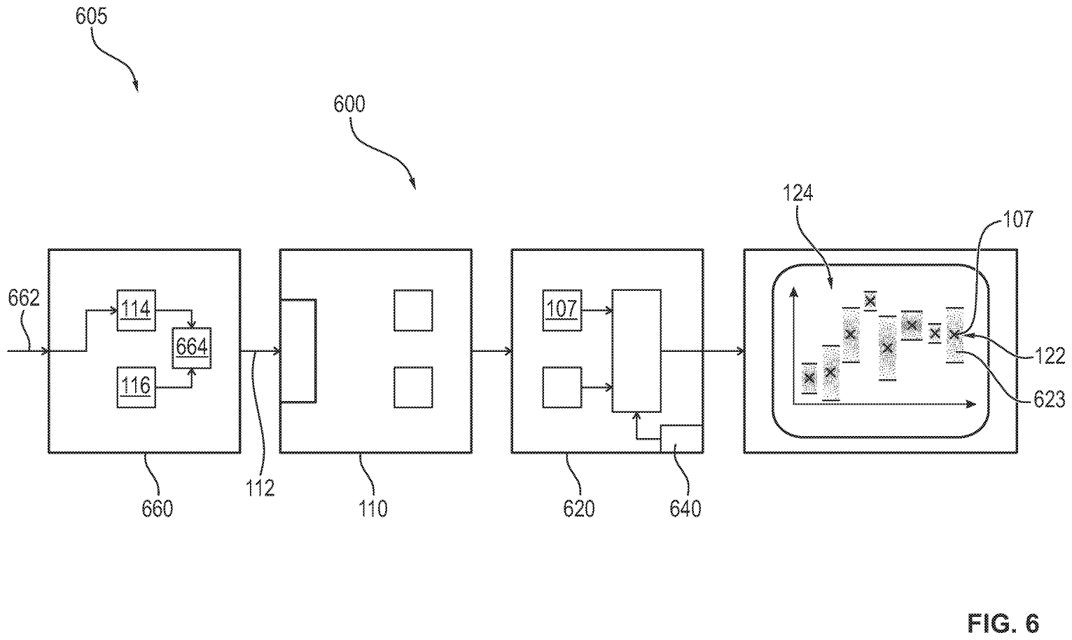

FIG. 6 shows a schematic view of a first exemplary embodiment of an output system 605 according to a second aspect of the present invention.

The output system 605 is configured to output the temporal course of the measured values 122 of a medical measured value 107. It comprises for this an output device 600 corresponding to the first aspect of the present invention and a data acquisition device 660.

The data acquisition device 660 is configured to detect the sensor data 114 via a received sensor signal 662, to determine the signal quality of the sensor signal 662, and to associate the corresponding signal quality data 116, especially the corresponding current signal quality data, with the sensor data 114. The sensor data 114 is combined in the exemplary embodiment shown with the correspondingly associated signal quality data 116 into a combined data set 664, which is then outputted. The combined data set 662 may be outputted to a network, to a patient data management system or the like. The received signal 112 received by the receiving unit 110 is made available directly by the data acquisition device 660 as an output in the exemplary embodiment shown.

The memory module 640 is not a separate module but is a part of the output control unit 620 in the exemplary embodiment shown.

The graph 124 shown shows discrete measured values 107, with a corresponding discrete curve spread 623. The curve spread 623 shows a course of a high-contrast center in the area of the measured values 107 towards a low-contrast edge area of this spread.

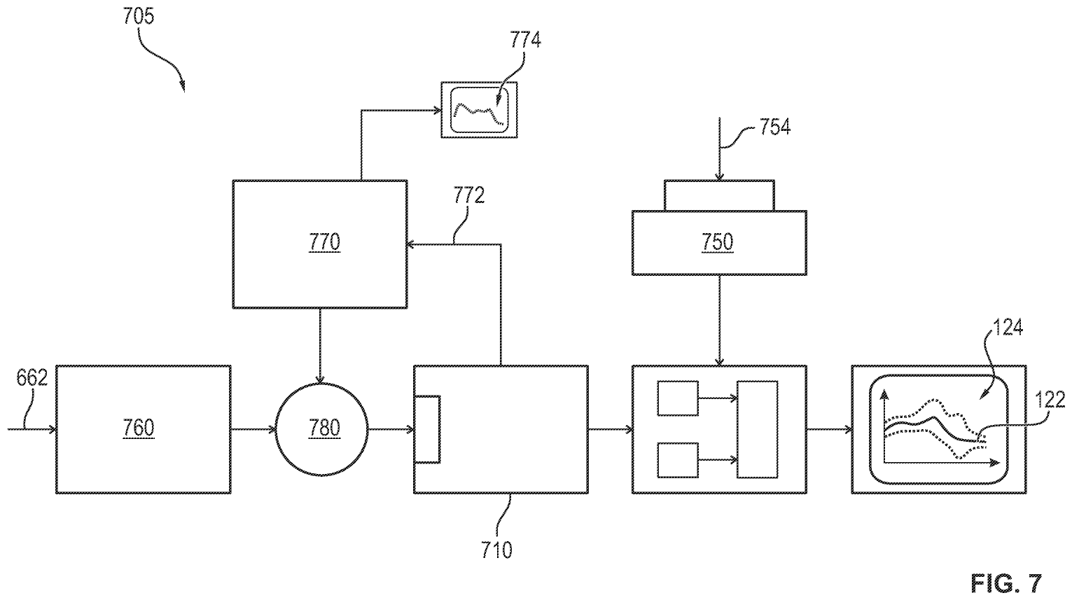

FIG. 7 shows a schematic view of a second exemplary embodiment of the output system 705 according to the second aspect of the present invention.

The output system 705 differs from the output system 605 shown in FIG. 6 in that it has a communication connection with an external alarm generation system 770. The communication connection takes place in this case via a network 780, via which the data acquisition unit 760 is connected to the receiving unit 710. In addition, the communication connection is formed by a back channel, via which the receiving unit 710 can output a warning signal 772 if a currently determined signal quality of the sensor signal 662 is below a predefined limit value. In an alternative or additional exemplary embodiment, the data acquisition device is configured to output such a warning signal to an external alarm generation system. In another alternative or additional exemplary embodiment, the output control unit is configured to output such a warning signal to an external alarm generation system.

The external alarm generation system 770 comprises an optical output 774, by which the undershooting of a predefined limit value for the currently determined signal quality is indicated.

Finally, the output system 705 comprises an input unit 750, via which a user input 554 can be received and subjected to further processing, as it was already described for the input unit 550. The user input 554 pertains in this case to an operating mode of the output system 705, to a selection of a graphic display for the graph 124 to the outputted, especially the spread of the temporal course of the measured values 122 to be displayed. from a stored plurality of graphic displays and/or a selection of a determination rule for the determination of the signal quality from a stored plurality of determination rules.

As an alternative or in addition, a duration of a past time range, which is to be displayed together with the graph to be outputted, can be set via the user input.

The determined signal quality is preferably determined on the basis of the signal-to-noise ratio of the received sensor signal 662 in the exemplary embodiment shown.

The graph 124 is a graph with an asymmetric curve spread around the temporal course of the measured values 122. The graphic display is carried out here corresponding to the exemplary embodiment shown in FIG. 1.

In one exemplary embodiment, not shown, the data acquisition device is configured, in addition, to determine and to output a correlation between the sensor data and the associated signal quality data over time, wherein the spread of the measured value curve is preferably determined on the basis of this correlation.

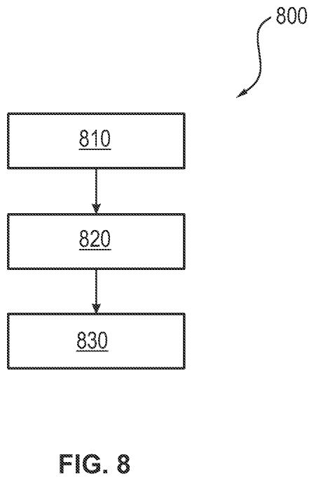

FIG. 8 shows a flow chart of an exemplary embodiment of a process 800 according to a third aspect of the present invention.

The process 800 according to the present invention is configured for outputting a temporal course of the measured values of a medical measured value. The process 800 has the steps explained below.

A first step 810 comprises a receipt and a provision of sensor data and signal quality data, wherein the sensor data indicate the temporal course of the measured values of the medical measured value, and wherein the signal quality data indicate a signal quality time curve associated with the temporal course of the measured values.

A next step 820 comprises a determination of a graph to be outputted with a measured value curve on the basis of the sensor data and the signal quality data and a provision of a corresponding output signal, wherein the measured value indicates the temporal course of the measured values and a curve spread of the temporal course of the measured values, and wherein the measured value curve indicates by its position and structure within the graph to be outputted the temporal course of the measured values of the medical measured value, and wherein the respective curve spread associated with a respective time of the measured value curve is a quality value indicative of the signal quality at this time of the measured value curve corresponding to the signal quality data.

Finally, a final step 830 comprises a receipt of the output signal and a graphic output of the graph to be outputted with the measured value curve, wherein the curve spread is displayed as a corresponding spread of the temporal course of the measured values.

Steps 810, 820 and 830 are carried out in the order shown. Thus, the received signal is always received first in order to control the output correspondingly and to finally output it in the final step 830.

The output corresponding to step 830 may take place now simultaneously with a repeated receipt of sensor data and signal quality data corresponding to step 810. For example, the process according to the present invention is thus repeated in time increments carried out at short intervals one after another in order to always display to current measured value curve.

Less than 10 sec, preferably less than 5 sec, and especially preferably less than 2 sec elapse between the receipt of the received signal in step 810 and the outputting of the output signal in step 830.

As is described in the exemplary embodiments shown, the process according to the present invention may be carried out for a continuous measured value curve to be displayed, which requires that the process 800 according to the present invention be carried out preferably especially rapidly. As an alternative or in addition, the process according to the present invention may be carried out for a discrete display of measured values, which requires a measurement in discrete time increments. The process is preferably carried out in this case in shorter time increments one after another than the time interval between two displayed measured values.

The process steps may be carried out by a single device, especially by a single processor. As an alternative, at least one of the process steps may be carried out at a location that is located at a distance from a location at which another one of these process steps is carried out. In particular, the process according to the present invention may be carried out by a plurality of processors.

While specific embodiments of the invention have been shown and described in detail to illustrate the application of the principles of the invention, it will be understood that the invention may be embodied otherwise without departing from such principles.

### LIST OF REFERENCE NUMBERS

100, 500, 600 Output device
102 Housing
107 Medical measured value
108 Signal quality
110, 510, 710 Receiving unit
111 Receiving interface
112, 512, 512′ Received signal
114 Sensor data
116 Signal quality data
118 Processed signal
120, 520, 620 Output control unit p0 122 Temporal course of the measured values
123, 223, 423, 523, 623 Curve spread
124 Graph to be outputted
125, 225, 325, 425 Measured value curve
126 Spread
128, 528 Output signal
130 Display unit
132 Display
509 Alarm generation limit
513 Alarm generation signal
517 Alarm generation data
527 Alarm generation line
540, 640 Memory module
550, 750 Input unit
552 Input interface
554, 754 User input
556 Input signal
605,705 Output system
660, 760 Data acquisition device
662 Sensor signal
664 Combined data set
770 External alarm generation system
772 Warning signal
774 Optical output
780 Network
800 Process
810, 820, 830 Process steps

What is claimed is:

1. An output device for outputting a measured value progression of a medical measured value, the output device comprising:
a receiving unit configured to receive sensor data and signal quality data via at least one received signal and to make the sensor data and the signal quality data available for further processing by the output device, wherein the sensor data indicate a temporal course of the measured values of the medical measured value, and wherein the signal quality data indicate a signal quality time curve associated with the temporal course of the measured values;
an output control unit configured to determine a graph to be outputted with a measured value curve based on the sensor data and of the signal quality data and to make the measured value curve available via an output signal, wherein the measured value curve indicates the temporal course of the measured values and a curve spread of the temporal course of the measured values, and wherein the measured value curve indicates, by a position and a structure within the graph to be outputted, the temporal course of the measured values of the medical measured value, and wherein the curve spread associated with a respective time of the measured value curve is a quality value indicative of the signal quality present at the respective time of the measured value curve corresponding to the signal quality data; and
a display unit comprising a display, wherein the display unit is configured to receive the output signal and to graphically output on the display the graph to be outputted with the measured value curve and with the corresponding curve spread, wherein the curve spread is displayed as a corresponding spread of the temporal course of the measured values.

2. An output device in accordance with claim 1, wherein the spread is a symmetrical spread of the temporal course of the measured values around a measured value indicated by the sensor data as the center of the spread.

3. An output device in accordance with claim 1, wherein areas of the measured value curve, which areas indicate the temporal course of the measured values, are displayed with higher contrast than the corresponding spread.

4. An output device in accordance with claim 1, wherein the spread is displayed as a continuously extending spread around the areas of the measured value curve, which area indicates the temporal course of the measured values.

5. An output device in accordance with claim 1, wherein the spread is inversely proportional to a signal quality present at the respective time corresponding to the signal quality data.

6. An output device in accordance with claim 1, wherein the output control unit is further configured to access a memory module with a stored plurality of graphic displays for the graph to be outputted.

7. An output device in accordance with claim 1, wherein the receiving unit is further configured to receive alarm generation data, wherein the alarm generation data indicate at least one alarm generation limit for the medical measured value, and wherein the output control unit is further configured to determine the graph to be outputted with the at least one alarm generation limit and to make the graph to be outputted with the at least one alarm generation limit available via the output signal.

8. An output system for outputting a measured value progression of a medical measured value, the output system comprising:
a data acquisition device configured to detect sensor data via a received sensor signal, to determine a signal quality of the sensor signal, to associate corresponding signal quality data with the sensor data and to output the sensor data and the associated signal quality data; and
an output device for outputting a measured value progression of a medical measured value, the output device comprising:
a receiving unit configured to receive the sensor data and the associated signal quality data from the data acquisition device via at least one received signal and to make the sensor data and the signal quality data available for further processing by the output device, wherein the sensor data indicate a temporal course of the measured values of the medical measured value, and wherein the signal quality data indicate a signal quality time curve associated with the temporal course of the measured values;
an output control unit configured to determine a graph to be outputted with a measured value curve based on the sensor data and the signal quality data and to make the measured value curve available via an output signal, wherein the measured value curve indicates the temporal course of the measured values and a curve spread of the temporal course of the measured values, and wherein the measured value curve indicates, by a position and a structure within the graph to be outputted, the temporal course of the measured values of the medical measured value, and wherein the curve spread associated with a respective time of the measured value curve is a quality value indicative of the signal quality present at the respective time of the measured value curve corresponding to the signal quality data; and
a display unit comprising a display, wherein the display unit is configured to receive the output signal and to graphically output on the display the graph to be outputted with the measured value curve and with the corresponding curve spread, wherein the curve spread is displayed as a corresponding spread of the temporal course of the measured values

9. An output system in accordance with claim 8, wherein:
the data acquisition device is configured to determine a correlation between the sensor data and the associated signal quality data over time and to output the correlation between the sensor data and the associated signal quality data over time to the output device; and
the output device is further configured to determine the spread of the measured value curve based on the correlation.

10. An output system in accordance with claim 8, further comprising a communication connection with an external alarm generation system, the communication connection providing a warning signal if a currently determined signal quality of the sensor signal is below a predefined limit value.

11. An output system in accordance with claim 8, further comprising an input interface, which is configured to receive a user input, wherein the user input pertains to at least one of:
an operating mode of the output system;
a selection of a graphic display for the graph to be outputted including the spread of the temporal course of the measured values, which spread is to be displayed, from a stored plurality of graphic displays and/or to a selection of a determination rules for the determination of the signal quality from a stored plurality of determination rules.

12. An output system in accordance with claim 8, wherein the determined signal quality is based on a signal-to-noise ratio of the received sensor signal.

13. An output system in accordance with claim 8, wherein the spread is a symmetrical spread of the temporal course of the measured values around a measured value indicated by the sensor data as the center of the spread.

14. An output system in accordance with claim 8, wherein areas of the measured value curve, which areas indicate the temporal course of the measured values, are displayed with higher contrast than the corresponding spread.

15. An output system in accordance with claim 8, wherein the spread is displayed as a continuously extending spread around the areas of the measured value curve, which area indicates the temporal course of the measured values.

16. An output system in accordance with claim 8, wherein the spread is inversely proportional to a signal quality present at the respective time corresponding to the signal quality data.

17. An output system in accordance with claim 8, wherein the output control unit is further configured to access a memory module with a stored plurality of graphic displays for the graph to be outputted.

18. An output system in accordance with claim 8, wherein the receiving unit is further configured to receive alarm generation data, wherein the alarm generation data indicate at least one alarm generation limit for the medical measured value, and wherein the output control unit is further configured to determine the graph to be outputted with the at least one alarm generation limit and to make the graph to be outputted with the at least one alarm generation limit available via the output signal.

19. A process for outputting a temporal course of the measured values of a medical measured value, the process comprising the steps of:
receiving and providing sensor data and signal quality data, wherein the sensor data indicate the temporal course of the measured values of the medical measured value, and wherein the signal quality data indicate a signal quality time curve associated with the temporal course of the measured values;
determining a graph to be outputted with a measured value curve based on the sensor data and of the signal quality data and providing a corresponding output signal, wherein the measured value curve indicates the temporal course of the measured values and a curve spread of the temporal course of the measured values, and wherein the measured value curve indicates the temporal course of the measured values of the medical measured value by a position and structure within the graph to be outputted, and wherein the curve spread associated with a respective time of the measured value curve is a quality value indicative of a signal quality present at this time of the measured value curve corresponding to the signal quality data (116); and
receiving the output signal and graphic outputting of the graph to be outputted with the measured value curve, wherein the curve spread is displayed as a corresponding spread of the temporal course of the measured values.

20. A process according to claim 19, wherein a computer program is provided with a program code for carrying out at least some of the process steps when the program code is executed on a computer, on a processor or on a programmable hardware component.
